(19) United States
(12) Patent Application Publication
(10) Pub. No.: US 2010/0099962 A1
(43) Pub. Date: Apr. 22, 2010

(54) SENSING DEVICE AND POSITIONING STRUCTURE THEREOF

(75) Inventors: Yung-Ming Chung, Changhua County (TW); Yi-Chin Tsai, Taipei County (TW); Shih-Chieh Yen, Taipei County (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: QUANTA COMPUTER INC., Tao Yuan Shien (TW)

(21) Appl. No.: 12/333,367

(22) Filed: Dec. 12, 2008

(30) Foreign Application Priority Data
Oct. 21, 2008 (TW) 97218778

(51) Int. Cl. A61B 5/1455; A61B 5/145

(52) U.S. Cl. 600/323; 600/322; 600/309

(57) ABSTRACT

A sensing device for sensing blood of a finger includes a base, a transceiver, a first positioning element and a second positioning element. The base includes a first half portion and a second half portion. The transceiver includes a first transmission element disposed on the first half portion, a second transmission element disposed on the second half portion, and a signal transmitted from one of the first and second transmission elements to the other. The first and second positioning elements are respectively disposed on the first and second half portions. The second positioning element is detachably engaged to the first positioning element. When the finger is disposed between the first and second half portions and the first and second positioning elements are engaged, the finger is located between the first and second transmission elements, and signals transmitted between the first and second transmission elements passes through blood of the finger.

### CROSS REFERENCE TO RELATED APPLICATIONS

This Application claims priority of Taiwan Patent Application No. 97218778, filed on Oct. 21, 2008, the entirety of which is incorporated by reference herein.

### BACKGROUND OF THE INVENTION

1. Field of the Invention

The present invention relates to a sensing device, and in particular relates to a sensing device and a positioning structure capable of detecting blood oxygen-levels.

2. Description of the Related Art

Generally, an important value for determining the cardiorespiratory function of a human body is the blood oxygen-level value (SpO₂). The ability to carry oxygen in the blood depends on the heart. If the heart or chest operates abnormally, the blood oxygen-level in the blood will fluctuate or decrease. Thus, the blood oxygen-level value (SpO₂) is not only provided to monitor a patient's vital signs, but also converted into related data as medical treatment reference for determining the precise amount of oxygen to supply to the patient.

A pulse oximeter is a common blood oxygen-level meter. During operation, a pulse oximeter probe is first fixedly adhered to a patient's lobe or finger. Following, a probe emitter generates light beams (with red light of 660 nm and infrared light of 940 nm), which are projected into a tissue area of the patient's lobe or finger. Next, a portion of the light beams are absorbed by the tissue of the lobe or the finger, and the remaining portion of the light beams pass through the tissue area and are received by a receiver of the probe. The intensity of the light beams that are received by the receiver is electronically converted into signals. After the signals are modulated, amplified and processed by a pulse oximeter circuit, the processed signals are transmitted to a microprocessor for calculation according to an average predetermined sample frequency, thereby resulting in the blood oxygen-level value. Then, the blood oxygen-level value is shown on a screen (e.g., LCD panel) or transmitted to a single grounded DC amplifier check instrument. Therefore, the accuracy of the blood oxygen-level value is fully determined by the probe.

Meanwhile, the external structure of a traditional blood oxygen-level instrument is generally made of rigid material. Additionally, a spring force applied so that the traditional blood oxygen-level instrument probe can be adhered to a patient's lobe or finger is usually uncomfortable for the patent.

### BRIEF SUMMARY OF THE INVENTION

In view of the above problems, the invention provides a foldable sensing device made of flexible material, thereby increasing the comfort level of a patients tested lobe or finger, while precisely detecting the blood oxygen-level value of the patient.

The invention provides a positioning structure for positioning a tested object with respect to a first transmission element and a second transmission element corresponding to the first transmission element. The positioning structure comprises a base, a first positioning element and a second positioning element.

The base comprises a first half portion and a second half portion. The first transmission element and the second transmission element are disposed on the first and second half portions, respectively. The first positioning element is disposed on the first half portion of the base. The second positioning element is disposed on the second half portion of the base and detachably connected to the first positioning element. When the tested object is disposed between the first and second half portions of the base, and the first positioning element disposed on the first half portion of the base and the second positioning element disposed on the second half portion of the base are connected, the first transmission element and the second transmission element are aligned and the tested object is located between the aligned first and second transmission elements.

The invention further provides a sensing device for sensing a content of a tested object. The sensing device comprises a base, a transceiver, a first positioning element and a second positioning element.

The base comprises a first half portion and a second half portion. The transceiver comprises a first transmission element and a second transmission element disposed on the first and second half portions of the base, respectively. A signal is transmitted from one of the first and second transmission elements to the other. The first positioning element is disposed on the first half portion of the base. The second positioning element is disposed on the second half portion of the base and detachably connected to the first positioning element. When the tested object is disposed between the first and second half portions of the base and the first positioning element disposed on the first half portion of the base and the second positioning element disposed on the second half portion of the base are connected, the first transmission element and the second transmission element are aligned and the tested object is located between the aligned first and second transmission elements, and the signal transmitted between the first and second transmission elements passes through the tested object.

With the foldable and flexible sensing device, the first transmission element and the second transmission element can be correctly aligned to increase precision of measuring the blood oxygen-level value, without sacrificing patient comfort like with the conventional art.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 is a schematic view of a sensing device of the invention;

FIG. 2A is a schematic view of a tested object disposed onto the sensing device of the invention;

FIG. 2B is a schematic view of the tested object partially enclosed by the sensing device of FIG. 2A;

FIG. 3 is a side view of the tested object partially enclosed by the sensing device of FIG. 2B; and

FIG. 4 is a schematic view showing the sensing device electronically connected to a system.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

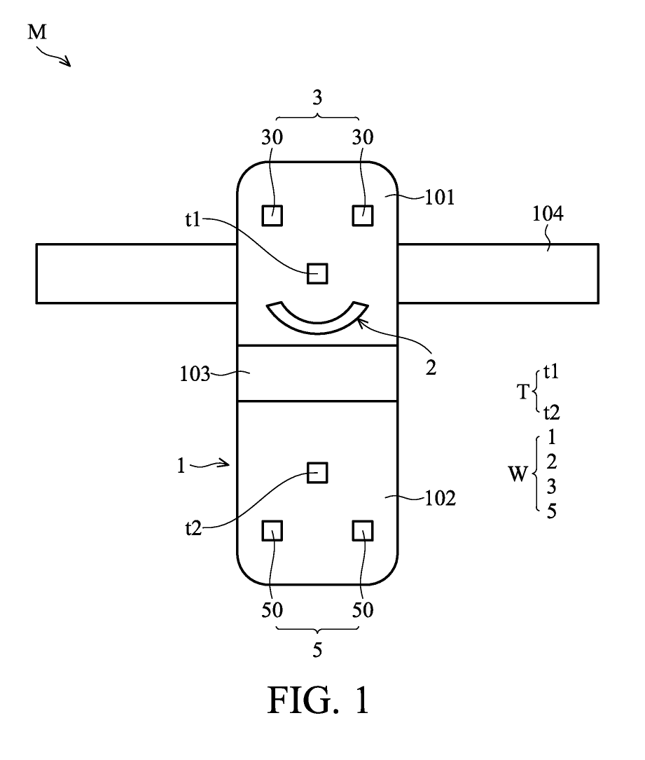

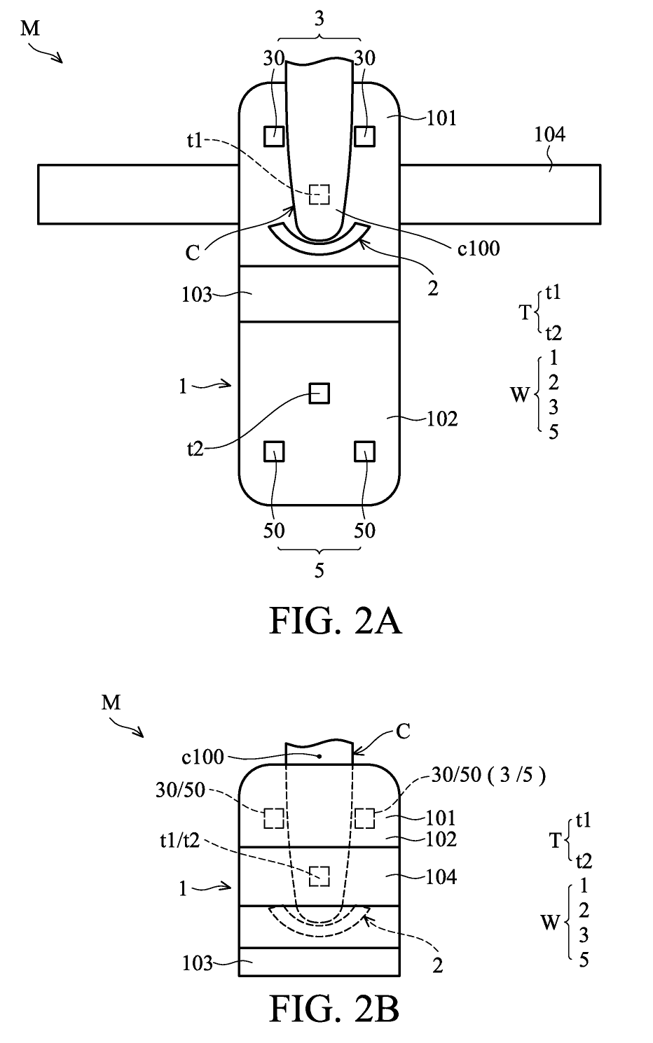

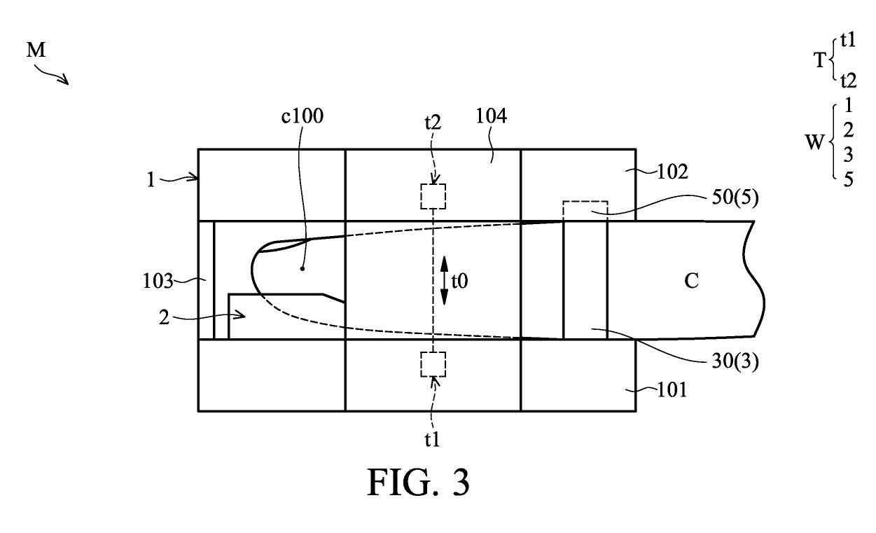

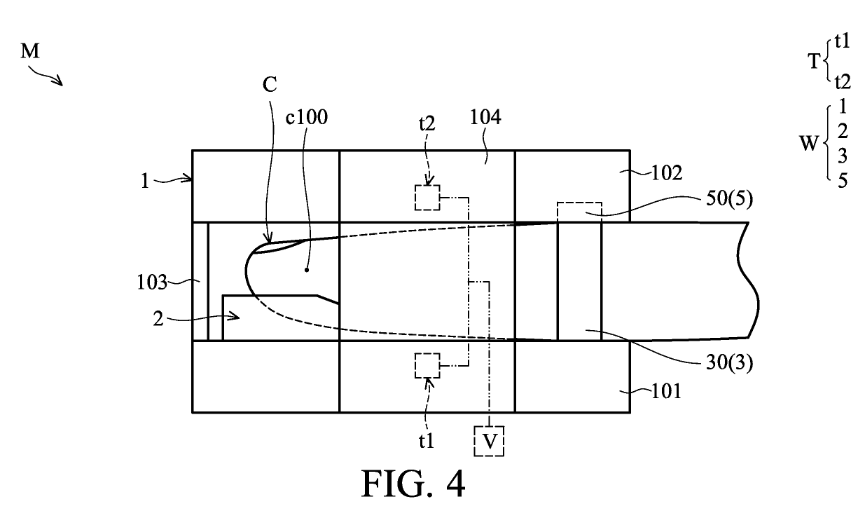

FIG. 1 is a schematic view of a sensing device M and a positioning structure W thereof of an embodiment, FIG. 2A is a schematic view of a tested object C disposed onto the sensing device M, FIG. 2B is a schematic view of the tested object C partially enclosed by the sensing device M of FIG. 2A, FIG. 3 is a side view of the tested object C partially enclosed by the sensing device M of FIG. 2B, and FIG. 4 is a schematic view showing the sensing device M electronically connected to a system V (e.g., a computer).

Referring to FIGS. 1, 2A and 2B, the sensing device M is utilized for sensing the property of a content c100 of a tested object C. In this embodiment, the tested object C is a finger, the content c100 is blood inside of the finger, and the property of blood comprises a blood oxygen-level. In the following description, the terms such as tested object C and the content c100 are respectively replaced by the finger and the blood.

The sensing device M comprises a base 1, a stopping element 2, a transceiver T having a first transmission element t1 and a second transmission element t2, a first positioning element 3, and a second positioning element 5. The base 1, the stopping element 2, the first positioning element 3 and the second positioning element 5 form the positioning structure W. With the positioning structure W, the finger C is positioned with respect to the first and second transmission elements t1 and t2 of the transceiver T.

The base 1 comprises a first half portion 101, a second half portion 102, a first connecting portion 103 disposed between the first and second half portions 101 and 102, and a second connecting portion 104 disposed on two sides of the first half portion 101. In this embodiment, the first and second half portions 101 and 102 of the base 1 are made of a flexible or rigid material, or other flexible material characterized with biocompatibility (e.g. organic material or the like). The first connecting portion 103 is made of a flexible or other flexible material characterized with biocompatibility. The second connecting portion 104 is made of a flexible belt-like material.

The stopping element 2 disposed on the base 1 is utilized to position the tested object C on the base 1 when the tested object C is stopped by the stopping element 2. In this embodiment, the stopping element 2 is a C shaped-like protrusion extended from the first half portion 101 of the base 1.

Referring to FIGS. 3 and 4, the first and second transmission elements t1 and t2 of the transceiver T correspond to each other to be respectively disposed on the first and second half portions 101 and 102 of the base 1 and electrically connect to the system V. A signal t0 is transmitted between the first and second transmission elements t1 and t2 of the transceiver T, wherein the first transmission element t1 serves as an emitter and the second transmission element t2 serves as a receiver. In this embodiment, the first transmission element t1 is a light-emitting diode which serves as an emitting terminal for emitting the signal t0, and the second transmission element t2 is a light detector which serves as a receiving terminal for receiving the signal t0 that passes through the tissue of the finger C.

The first and second positioning elements 3 and 5 are respectively disposed on the first and second half portions 101 and 102 of the base 1, to be detachably connected to each other. In this embodiment, the first positioning element 3 comprises two spaced positioning posts 30, and the second positioning element 5 comprises two positioning holes 50 corresponding to and detachably connected to the positioning posts 30 of the first positioning element 3.

In FIG. 2A, when the first and second half portions 101 and 102 of the base 1 is horizontally placed and the finger C is externally placed between the positioning posts 30 of the first positioning element 3, the finger C is capable of moving toward the stopping element 2 (or along the direction pointing to the first connecting portion 103) by the guidance of the positioning posts 30. When the finger C is stopped by the stopping element 2, the finger C is properly positioned among the positioning posts 30 and the stopping element 2, and therefore the first transmission element t1 of the transceiver T is correctly located at one side of or below the finger C.

In FIGS. 2B and 3, when the second half portion 102 of the base 1 is relatively folded toward the first half portion 101 thereof by wrapping the finger C therebetween, the finger C is partially covered between the first and second half portions 101 and 102 of the base 1. Then, when the first positioning element 3 disposed on the first half portion 101 and the second positioning element 5 disposed on the second half portion 102 are connected, the first transmission element t1 and the second transmission element t2 are aligned and the tested object C is located between the aligned first and second transmission elements t2.

When the finger C is disposed between the first and second half portions 101 and 102 of the base 1 and the first positioning element 3 disposed on the first half portion 101 and the second positioning element 5 disposed on the second half portion 102 are connected, the connected first and second half portions 101 and 102 can be enclosed by the second connecting portion 104, thereby positioning the finger C tightly between the first half portion 101 and the second half portion 102 and decreasing physical vibration from finger C.

When the signal t0 transmitted between the first and second transmission elements t1 and t2 passes through the blood c100 of the finger C, the portion of the signal t0 which passes through the blood c100 of the finger C received by the second transmission element t2 is transmitted to the system V. After conversion and calculation by the system V, the oxygen-level of the blood c100 of the finger C is obtained.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A positioning structure utilized for positioning a tested object with respect to a first transmission element and a second transmission element corresponding to the first transmission element, comprising:
a base comprising a first half portion and a second half portion, wherein the first transmission element and the second transmission element are disposed on the first and second half portions, respectively;
a first positioning element disposed on the first half portion of the base; and
a second positioning element disposed on the second half portion of the base and detachably connected to the first positioning element,
wherein when the tested object is disposed between the first and second half portions of the base and the first positioning element disposed on the first half portion of the base and the second positioning element disposed on the second half portion of the base are connected, the first transmission element and the second transmission element are aligned and the tested object is located between the aligned first and second transmission elements.

2. The positioning structure as claimed in claim 1, further comprising a stopping element disposed on the base, wherein the tested object is stopped by the stopping element and positioned on the base.

3. The positioning structure as claimed in claim 2, wherein the stopping element comprises a C shaped-like protrusion.

4. The positioning structure as claimed in claim 1, wherein the base further comprises a first connecting portion disposed between the first and second half portions.

5. The positioning structure as claimed in claim 4, wherein the first connecting portion of the base comprises a flexible material.

6. The positioning structure as claimed in claim 1, wherein the base further comprises a second connecting portion utilized to enclose the first and second half portions when the tested object is disposed between the first and second half portions and the first positioning element disposed on the first half portion of the base and the second positioning element disposed on the second half portion of the base are connected.

7. The positioning structure as claimed in claim 6, wherein the second connecting portion of the base comprises a flexible material.

8. The positioning structure as claimed in claim 1, wherein the first and second half portions of the base comprises a flexible or rigid material.

9. A sensing device utilized for sensing a content of a tested object, comprising:
a base comprising a first half portion and a second half portion;
a transceiver comprising a first transmission element and a second transmission element disposed on the first and second half portions of the base, respectively, wherein a signal is transmitted from one of the first and second transmission elements to the other;
a first positioning element disposed on the first half portion of the base; and
a second positioning element disposed on the second half portion of the base and detachably connected to the first positioning element,
wherein when the tested object is disposed between the first and second half portions of the base and the first positioning element disposed on the first half portion of the base and the second positioning element disposed on the second half portion of the base are connected, the first transmission element and the second transmission element are aligned and the tested object is located between the aligned first and second transmission elements, and the signal transmitted between the first and second transmission elements passes through the content of the tested object.

10. The sensing device as claimed in claim 9, further comprising a stopping element disposed on the base, wherein the tested object is stopped by the stopping element and positioned on the base.

11. The sensing device as claimed in claim 10, wherein the stopping element comprises a C shaped-like protrusion.

12. The sensing device as claimed in claim 9, wherein the base further comprises a first connecting portion disposed between the first and second half portions.

13. The sensing device as claimed in claim 12, wherein the first connecting portion of the base comprises a flexible material.

14. The sensing device as claimed in claim 9, wherein the base further comprises a second connecting portion utilized to enclose the first and second half portions when the tested object is disposed between the first and second half portions and the first positioning element disposed on the first half portion of the base and the second positioning element disposed on the second half portion of the base are connected.

15. The sensing device as claimed in claim 14, wherein the second connecting portion of the base comprises a flexible material.

16. The sensing device as claimed in claim 9, wherein the first and second half portions of the base comprises a flexible or rigid material.

17. The sensing device as claimed in claim 16, wherein the flexible material is characterized with biocompatibility.

18. The sensing device as claimed in claim 9, wherein the first transmission element comprises an emitter.

19. The sensing device as claimed in claim 18, wherein the second transmission element comprises a receiver.

20. The sensing device as claimed in claim 9, wherein the tested object comprises a finger and the content comprises blood of the finger.
